(19) United States
(12) Patent Application Publication
(10) Pub. No.: US 2014/0193525 A1
(43) Pub. Date: Jul. 10, 2014

(54) METHODS FOR TREATING OR PREVENTING RADIOCONTRAST AGENT INDUCED KIDNEY INJURY

(71) Applicant: Fred Hutchinson Cancer Research Center, Seattle, WA (US)

(72) Inventors: Chienmin Su, Redmond, WA (US); Csaba Szabo, Seattle, WA (US)

(73) Assignee: Fred Hutchinson Cancer Research Center, Seattle, WA (US)

(21) Appl. No.: 13/968,163

(22) Filed: Aug. 15, 2013

Related U.S. Application Data
(63) Continuation of application No. 12/690,368, filed on Jan. 20, 2010, now abandoned.
(60) Provisional application No. 61/146,222, filed on Jan. 21, 2009.

(51) Int. Cl. A61K 33/04

(52) U.S. Cl. CPC A61K 33/04
USPC 424/706; 424/703; 424/709; 424/711

(57) ABSTRACT

Methods for treating or preventing radiocontrast agent induced kidney injury in a mammal are disclosed, the methods comprising administering to the mammal a first effective amount of a chalcogenide composition prior to administering a radiocontrast agent to the mammal.

### CROSS REFERENCE TO RELATED APPLICATIONS

This application is a continuation of U.S. patent application Ser. No. 12/690,368, filed Jan. 20, 2010, which claims the benefit under 35 USC 119(e) to U.S. Provisional Application Ser. No. 61/146,222, filed Jan. 21, 2009, entitled “Methods for Treating or Preventing Radiocontrast Agent Induced Kidney Injury”. Each of these applications is hereby incorporated by reference in its entirety.

### BACKGROUND

Radiocontrast agent induced kidney injury represents a significant clinical problem and has been associated with increased patient mortality and longer hospitalizations. Radiocontrast agent administration, conducted in conjunction with imaging of the coronary arteries, the peripheral arteries or the kidneys is a common medical intervention. However, radiocontrast agent administration often leads to significant kidney damage in a subset of patients with certain risk factors. For example, patients who are elderly, have an underlying disease (e.g., diabetes or atherosclerosis) and/or have a borderline impairment in their renal function are especially predisposed to kidney injury.

The mechanism of radiocontrast agent induced kidney injury is multiple, and is believed to have oxidative, inflammatory and vascular components. To date, the primary strategy for prevention of radiocontrast agent induced kidney injury is hydration with intravenous fluids. In addition to hydration, the use of various pharmaceuticals have been studied but none has been definitively proven or accepted as beneficial in preventing radiocontrast agent induced kidney injury. To date, the most studied pharmaceutical is N-acetylcysteine (NAC). While some studies have shown NAC to be effective in reducing serum creatinine, the data is mixed and the consensus appears to be that its effectiveness in preventing radiocontrast agent induced kidney injury is limited (see, e.g., Drager, L. F. et al., Nephrol. Dial. Transplant 19: 1803-1807 (2004)). Other pharmaceuticals that have been studied include ascorbic acid, theophylline, fenoldopam, calcium antagonists, and periprocedural hemofiltration (see, e.g., Itoh, Y. et al., J. Pharmacol. Sci. 97: 473-488 (2005)). However, data for these therapies are also mixed. For example, while in one study periprocedural hemofiltration appeared to be effective in preventing radiocontrast agent induced kidney injury in the ICU setting, it was noted that this procedure is invasive and expensive and not directly applicable to all high-risk patients exposed to contrast agents.

Accordingly, while progress has been made in this field, there remains a need in the art for methods for treating or preventing radiocontrast agent induced kidney injury and pharmaceutical compositions useful for the same. The present invention fulfills this need and provides further related advantages.

### SUMMARY OF THE INVENTION

One aspect of the invention is a method for treating or preventing radiocontrast agent induced kidney injury in a mammal (e.g., a human patient in need thereof) comprising administering to the mammal a first effective amount (i.e., a dose) of HS⁻. The HS⁻ is an active pharmaceutical ingredient (API). The first effective amount of HS⁻ may be administered to the patient in a first stable aqueous pharmaceutical composition comprising the first effective amount of HS⁻ and deoxygenated water. The first stable aqueous pharmaceutical composition is administered prior to administration of the radiocontrast agent to the mammal.

In an exemplary embodiment of the method, the method further comprises administering a second effective amount of HS⁻ to the mammal during administration of the radiocontrast agent to the mammal. The second effective amount of HS⁻ may be administered to the patient in a second stable aqueous pharmaceutical composition comprising the second effective amount of HS⁻ and deoxygenated water.

In another exemplary embodiment of the method, the HS⁻ in the first and/or second stable aqueous pharmaceutical compositions are made by dissolving in water a chaldogenide selected from H₂S, Na₂S, NaHS, K₂S, KHS, Rb₂S, CS₂S, (NH₄)₂S, (NH₄)HS, BeS, MgS, CaS, SrS, BaS, H₂Se, Na₂Se, NaHSe, K₂Se, KHSe, Rb₂Se, CS₂Se, (NH₄)₂Se, (NH₄)HSe, BeSe, MgSe, CaSe, SrSe, PoSe, BaSe, or, a pharmaceutically acceptable salt thereof.

In another exemplary embodiment of the method, the HS⁻ in the first and/or second stable aqueous pharmaceutical compositions are made by dissolving NaHS in water.

In another exemplary embodiment of the method, the first and/or second stable aqueous pharmaceutical compositions comprises the HS⁻ and one or more pharmaceutically acceptable carriers, diluents and excipients.

In another exemplary embodiment of the method, administration of the first stable aqueous pharmaceutical composition is a bolus injection, and, administration of the second stable aqueous pharmaceutical composition is an intravenous infusion.

In another exemplary embodiment, the radiocontrast agent is administered before or during kidney diagnostic imaging.

In another exemplary embodiment, the radiocontrast agent is administered before or during heart blood vessel diagnostic imaging.

In another exemplary embodiment of the method, the radiocontrast agent is an iodinated radiocontrast agent.

In another exemplary embodiment of the method, the mammal (e.g., human patient in need of treatment thereof) has a pre-existing impairment of renal function prior to administration of the radiocontrast agent.

In another exemplary embodiment of the method, the human has a baseline creatinine level greater than or equal to 120 μmol/L, a glomerular filtration rate of less than 60 mL/min per 1.73 m² and/or a creatinine clearance of less than 60 mL/min.

In another exemplary embodiment of the method, the human has one of more conditions selected from type 1 or 2 diabetes mellitus, atherosclerosis, congestive heart failure, an intraarterial balloon pump, anemia, a systolic blood pressure of less than 80 mm Hg, an age greater than 50 years, a glomerular filtration rate of less than 60 mL/min per 1.73 m² and reduced intravascular volume.

In another exemplary embodiment of the method, the radiocontrast agent induced kidney injury is radiocontrast agent induced nephropathy or acute renal dysfunction.

In another exemplary embodiment of the method, the water is deoxygenated water having an O₂ content 5 μM.

In another exemplary embodiment of the method, the first and/or second effective amounts of HS⁻ are administered at a concentration in the range of 1 mM to 250 mM, or 10 mM to 200 mM.

In another exemplary embodiment of the method, the first and/or second stable aqueous pharmaceutical compositions have a pH in the range of 6.5-8.5, 7.0-9.0, 7.5-8.5, 7.4-9.0, or, 7.5-8.0. The pH may be adjusted by adding HCl or NaOH to the composition.

In another exemplary embodiment of the method, the first and/or second stable aqueous pharmaceutical compositions further comprises one or more oxidation products selected from polysulfide, sulfite, sulfate and thiosulfate.

In another exemplary embodiment of the method, the first and/or second stable aqueous pharmaceutical compositions comprise polysulfide in the range of 0-1.0%, sulfite in the range of 0-1.0%, sulfate in the range of 0-1.0%, and/or, thiosulfate in the range of 0-1.0%.

In another exemplary embodiment of the method, the first and/or second stable aqueous pharmaceutical composition has an osmolarity in the range of 250-330 mOsmol/L.

In another exemplary embodiment of the method, the mammal (e.g., human patient in need of treatment thereof) is contacted (e.g., treated) with the first and/or second stable aqueous pharmaceutical compositions before or after the kidney injury.

In another exemplary embodiment of the method, the first and/or second stable aqueous pharmaceutical compositions are near isotonic.

In another exemplary embodiment, a chalcogenide composition is made from hydrogen sulfide or a pharmaceutically acceptable salt thereof. For example, in certain embodiments, the chalcogenide composition is made from sodium sulfide.

These and other aspects of the invention will be apparent upon reference to the following detailed description.

### DESCRIPTION OF DRAWINGS

FIG. 1 is a graph showing blood urine nitrogen levels before and after administration of a liquid sulfide composition as described in Example 1.

FIG. 2 is a graph showing blood creatine levels before and after administration of a liquid sulfide composition as described in Example 1.

FIG. 3 is a graph showing blood glucose levels before and after administration of a liquid sulfide composition as described in Example 1.

### DETAILED DESCRIPTION OF EXEMPLARY EMBODIMENTS

In the following description, certain specific details are set forth in order to provide a thorough understanding of various embodiments of the invention. However, one skilled in the art will understand that the invention may be practiced without these details.

Unless the context requires otherwise, throughout the present specification and claims, the word “comprise” and variations thereof, such as, “comprises” and “comprising” are to be construed in an open, inclusive sense, that is as “including, but not limited to”.

Reference throughout this specification to “one embodiment” or “an embodiment” means that a particular feature, structure or characteristic described in connection with the embodiment is included in at least one embodiment of the present invention. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

“Chalcogenide” or “chalcogenide compounds” refers to compounds containing a chalcogen element, i.e., those in Group 6 of the periodic table, but excluding oxides. These elements are sulfur (S), selenium (Se), tellurium (Te) and polonium (Po). Specific chalcogenides and salts thereof include, but are not limited to: H₂S, Na₂S, NaHS, K₂S, KHS, Rb₂S, CS₂S, (NH₄)₂S, (NH₄)HS, BeS, MgS, CaS, SrS, BaS, H₂Se, Na₂Se, NaHSe, K₂Se, KHSe, Rb₂Se, CS₂Se, (NH₄)₂Se, (NH₄)HSe, BeSe, MgSe, CaSe, SrSe, PoSe and BaSe.

“Chalcogenide composition” refers to a composition containing one or more chalcogenides or chalcogenide compounds.

“Sulfide” refers to sulfur in its −2 valence state, either as H₂S or as a salt thereof (e.g., NaHS, Na₂S, etc.). Sulfide also refers to deuterium sulfide or ²HS. “H₂S” is generated by the spontaneous dissociation of the chalcogenide salt and H₂S donor, for example, sodium hydrosulfide (NaHS), in aqueous solution according to the equations:

NaHS→Na⁺+HS⁻

4HS⁻ [symbol] 2H₂S+S₂⁻

HS⁻+H⁺ [symbol] H₂S

It is well known in the art that sulfides are unstable compounds and produce oxidation products. As used herein, “sulfide oxidation product” refers to products that result from sulfide chemical transformation, including, e.g., sulfite, sulfate, thiosulfate, polysulfides, dithionate, polythionate, and elemental sulfur.

“Pharmaceutically acceptable salt” includes both acid and base addition salts. In certain embodiments, the chalcogenide composition comprises a pharmaceutically acceptable salt, preferably salts wherein the chalcogenide is in a −2 oxidation state. For example, sulfide salts encompassed by embodiments of the invention include, but are not limited to, sodium sulfide (Na₂S), sodium hydrogen sulfide (NaHS), potassium sulfide (K₂S), potassium hydrogen sulfide (KHS), lithium sulfide (Li₂S), rubidium sulfide (Rb₂S), cesium sulfide (Cs₂S), ammonium sulfide ((NH₄)₂S), ammonium hydrogen sulfide (NH₄)HS, beryllium sulfide (BeS), magnesium sulfide (MgS), calcium sulfide (CaS), strontium sulfide (SrS), barium sulfide (BaS), and the like.

“Pharmaceutical composition” refers to a formulation of a compound (or composition) and a medium generally accepted in the art for the delivery of the biologically active compound to mammals, e.g., humans. Such a medium includes all pharmaceutically acceptable carriers, diluents or excipients therefor.

“Pharmaceutically acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

“Radiocontrast agent induced kidney injury” refers to nephropathy induced by exposure to radiocontrast agents, which may range from a transient elevation of the serum creatinine concentration to acute renal failure. In one embodiment, a transient elevation of the serum creatinine concentration may be defined as a greater than 25% increase of serum creatinine concentration or an absolute increase in serum creatinine concentration of 0.5 mg/dL. Acute renal failure is generally characterized by a rapid fall in glomerular filtration rate, clinically manifest as an abrupt and sustained rise in urea and creatinine (see, e.g., Bellomo et al., Crit. Care 8(4): R204-R212 (2004), and Hilton R., BMJ 333(7572): 786-790 (2006)) which may occur over hours or days (Schrier, R. W. et al., J. Clin. Invest. 114(1): 5-14 (2004)).

“Radiocontrast agent” refers to a medical contrast medium used to improve the visibility of internal bodily structures in x-ray based imaging techniques such as computed tomography (CT) and radiography (commonly known as x-ray imaging). Radiocontrast agents are typically iodine or barium compounds, including, for example, the following commonly used iodinated radiocontrast agents: diatrizoate or diatrizoic acid (commercially available as, e.g., Hypaque), metrizoate (commercially available as, e.g., Isopaque), ioxaglate (commercially available as, e.g., Hexabrix), iopamidol (commercially available as, e.g., Isovue), iohexyl (commercially available as, e.g., Omnipaque), ioxilan (commercially available as, e.g., Oxilan), iopomide (commercially available as, e.g., Ultravist) and iodixanol (commercially available as, e.g., Visipaque).

“Preventing” or “prevention” as used herein covers preventing the disease or condition of interest (namely, radiocontrast agent induced kidney injury) from occurring in a mammal, in particular, when such mammal is predisposed to the condition but has not yet been diagnosed as having it.

“Treating” or “treatment” as used herein covers the treatment of the disease or condition of interest (namely, radiocontrast agent induced kidney injury) in a mammal having the disease or condition of interest, and includes, for example, inhibiting the disease or condition, i.e., arresting its development; relieving the disease or condition, i.e., causing regression of the disease or condition; or, relieving the symptoms resulting from the disease or condition, e.g., relieving pain without addressing the underlying disease or condition.

As used herein, the terms “disease” and “condition” may be used interchangeably or may be different in that the particular malady or condition may not have a known causative agent (so that etiology has not yet been worked out) and it is therefore not yet recognized as a disease but only as an undesirable condition or syndrome, wherein a more or less specific set of symptoms have been identified by clinicians.

“Administering” includes routes of administration which allow an agent (e.g., a chalcogenide composition) to perform its intended function, e.g., treating or preventing radiocontrast agent induced kidney injury. A variety of routes of administration are possible including, but not necessarily limited to parenteral (e.g., intravenous, intra-arterial, intramuscular, subcutaneous injection), oral (e.g., dietary), topical, nasal, inhalation, rectal, or via slow releasing micro-carriers.

“Effective amount” or “therapeutically effective amount” refers to that amount of an agent, e.g., a chalcogenide composition, that, when administered to a mammal, is sufficient to perform its intended function, e.g., treating or preventing radiocontrast agent induced kidney injury. The amount of an agent which constitutes an “effective amount” will depend upon a number of factors, including the agent, the manner of administration, and the condition of the mammal to be treated (e.g., age, any underlying diseases and/or impairments in renal function, etc. . . . ), but can be determined routinely by one of ordinary skill in the art having regard to his own knowledge and to this disclosure. It is also understood that an effective amount of an agent may be a different amount when the agent is used alone as compared to when it is used in combination with another agent.

“Mammal” includes humans and both domestic mammals, such as laboratory animals and household pets (e.g., cats, dogs, swine, cattle, sheep, goats, horses, rabbits), and non-domestic mammals, such as wildlife and the like.

As further described below, in certain embodiments, the chacogenide composition may be administered in a stable liquid pharmaceutical composition. “Liquid” as used with regard to pharmaceutical compositions is intended to include the term “aqueous.” “Stable” refers to the concentration of the active chalcogenide composition, the pH of the chalcogenide composition and/or oxidation products of the chalcogenide composition remaining within a range of “acceptance criteria” after storage of the liquid pharmaceutical composition for a pre-specified time period.

“Acceptance criteria” refers to the set of criteria to which a drug substance or drug product should conform to be considered acceptable for its intended use. As used herein, acceptance criteria are a list of tests, references to analytical procedures, and appropriate measures, which are defined for a drug product that will be used in a mammal. For example, the acceptance criteria for a stable liquid pharmaceutical composition of a chalcogenide composition refers to a set of predetermined ranges of drug substance concentration, pH, and levels of oxidation products that are acceptable for pharmaceutical use for the specific drug composition based on stability testing. Acceptance criteria may be different for other formulations, include those for topical and cosmetic use. Acceptable standards are generally defined for each industry. For example, various acceptance criteria include any value or range described herein that meets Good Manufacturing Practice Regulations promulgated by the U.S. Food and Drug Administration. In certain embodiments, an acceptance criteria is a pH in the range of 7.4 to 9.0, 6.5 to 8.5, or 6.5 to 9.0 at a time point of 0, 1, 2, 3, or 4 months storage at 4° C., 25° C., or 40° C. In certain embodiments, an acceptance criteria is an osmolality in a range of 250-350 mOsm/kg or an osmolarity in the range of 250-330 mOsm/L at a time point of 0, 1, 2, 3, or 4 months storage at 4° C., 25° C., or 40° C. In certain embodiments, an acceptance criteria is a sulfide concentration of 5.0-6.0 mg/ml at a time point of 0, 1, 2, 3, or 4 months storage at 4° C., 25° C., or 40° C. In certain embodiment, an acceptance criteria is a concentration of chalcogenide within the range of 0.1-100 mg/ml, 1-10 mg/ml, or 95-150 mM at a time point of 0, 1, 2, 3, or 4 months storage at 4° C., 25° C., or 40° C. In certain embodiments, an acceptance criteria is sulfide present at least 80%, at least 85%, at least 90%, at least 95%, at least 96%, at least 97%, at least 98%, or at least 99% weight/volume of total sulfide and oxidation products thereof at a time point of 0, 1, 2, 3, or 4 months storage at 4° C., 25° C., or 40° C. In certain embodiments, oxidation products are present at a concentration less than 10%, less than 5%, less than 4%, less than 3%, less than 2%, less than 1%, 0.5% or less of total sulfide and oxidation products at a time point of 0, 1, 2, 3, or 4 months storage at 4° C., 25° C., or 40° C.

“%” when used without qualification (as with w/v, v/v, or w/w) means % weight-in-volume for solutions of solids in liquids (w/v), % weight-in-volume for solutions of gases in liquids (w/v), % volume-in-volume for solutions of liquids in liquids (v/v) and weight-in-weight for mixtures of solids and semisolids (w/w) (Remington's Pharmaceutical Sciences (2005); 21^{st} Edition, Troy, David B. Ed. Lippincott, Williams and Wilkins).

As noted above, it has been discovered that chacogenide compositions, such as hydrogen sulfide and pharmaceutically acceptable salts thereof, may be utilized to treat or prevent radiocontrast agent induced kidney injury in a mammal. Previously, it has been shown that treatment with chalcogenides protects biological matter from hypoxic and ischemic injury. In these studies, it was demonstrated that a stable composition of hydrogen sulfide (H₂S), a potent inhibitor of oxygen consumption, reduced hypoxic injuries in small and large animals (see U.S. Patent Application Publication Nos. 2007/0078113, 2008/0187694 and 2008/0199541, which are incorporated herein by reference). Although hydrogen sulfide, as well as other chacogenide compositions, have not been typically considered for medical use, this unexpected result presented exciting possibilities for the treatment or prevention of a number of animal and human diseases, particularly hypoxia and ischemia-related diseases and injuries. However, to date, sulfide has not been shown to treat or prevent radiocontrast agent induced kidney injury.

In one embodiment, a method for treating or preventing radiocontrast agent induced kidney injury in a mammal is provided, the method comprising administering to the mammal a first effective amount of a chalcogenide composition prior to administering a radiocontrast agent to the mammal. Radiocontrast agents are commonly administered in conjunction with imaging of the coronary arteries, the peripheral arteries or the kidneys is a common medical intervention. Accordingly, in certain embodiments, the radiocontrast agent is being administered before or during kidney or heart blood vessel diagnostic imaging. In certain embodiments, the mammal is a human.

In certain embodiments, the chalcogenide composition comprises hydrogen sulfide or a pharmaceutically acceptable salt thereof. For example, in certain specific embodiments, the chalcogenide composition comprises sodium sulfide. While specific embodiments of the present invention described herein are directed to sulfur compounds, it is understood that in other embodiments, the present invention may be practiced using chalcogenides other than sulfur. In certain embodiments, the chalcogenide compound comprises sulfur, while in others it comprises selenium, tellurium, or polonium.

In further embodiments, the method further comprises administering a second effective amount of the chalcogenide composition to the mammal during administration of the radiocontrast agent to the mammal. For example, the chalcogenide composition may be administered as a bolus injection followed by an intravenous infusion.

While the risk of radiocontrast agent induced kidney damage is low in the general population, the risk is greatly increased in patients with certain risk factors. Accordingly, in further embodiments, the mammal has a pre-existing impairment of renal function prior to administration of the radiocontrast agent. For example, in certain embodiments, the mammal has a baseline creatinine level greater than or equal to 120 pmol/L, a glomerular filtration rate of less than 60 mL/min per 1.73 m² or a creatinine clearance of less than 60 mL/min. In other further embodiments, the mammal has one of more conditions selected from the group consisting of type 1 or 2 diabetes mellitus, atherosclerosis, congestive heart failure, an intraarterial balloon pump, anemia, a systolic blood pressure of less than 80 mm Hg, an age greater than 50 years, a glomerular filtration rate of less than 60 mL/min per 1.73 m² and reduced intravascular volume.

In addition, a variety of agents have previously been identified that may be used to treat or prevent radiocontrast agent induced kidney injury. Examples of such agents include, but are not limited to, N-acetylcysteine, ascorbic acid, theophylline, fenoldopam, calcium antagonists, and periprocedural hemofiltration. Accordingly, as one of skill in the art will appreciate, in certain embodiments the present invention provides a method for treating or preventing radiocontrast injured kidney damage in a mammal comprising administering to the mammal a first effective amount of a chalcogenide composition in combination with one or more of these other agents prior to administering a radiocontrast agent to the mammal. Furthermore, as one of skill in the art will appreciate, when a chalcogenide composition is used in combination with one or more of these other agents, the agents (i.e., the chalcogenide composition and the other agent) may be administered simultaneously or in any order. For example, the time periods during which the agents are administered may overlap or be distinct.

For the purposes of administration, a chalcogenide composition may be administered directly or may be formulated as a pharmaceutical composition. Pharmaceutical compositions of the present invention comprise a chalcogenide composition and a pharmaceutically acceptable carrier, diluent or excipient. The chalcogenide composition is present in the composition in an amount which is effective to treat or prevent radiocontrast agent induced kidney injury, and preferably, with acceptable toxicity.

Administration of a chalcogenide composition, in pure form or in an appropriate pharmaceutical composition, can be carried out via any of the accepted modes of administration of agents for serving similar utilities. The pharmaceutical compositions of the invention can be prepared by combining a chalcogenide composition with an appropriate pharmaceutically acceptable carrier, diluent or excipient, and may be formulated into preparations in solid, semi-solid, liquid or gaseous forms, such as tablets, capsules, powders, granules, ointments, solutions, suppositories, injections, inhalants, gels, microspheres, and aerosols. Typical routes of administering such pharmaceutical compositions include, without limitation, oral, topical, transdermal, inhalation, parenteral, sublingual, buccal, rectal, vaginal, and intranasal. The term parenteral as used herein includes subcutaneous injections, intravenous, intramuscular, intrasternal injection or infusion techniques. Pharmaceutical compositions of the invention are formulated so as to allow the active ingredients contained therein to be bioavailable upon administration of the composition to a patient. Compositions that will be administered to a subject or patient take the form of one or more dosage units, where for example, a tablet may be a single dosage unit, and a container of a compound of the invention in aerosol form may hold a plurality of dosage units. Actual methods of preparing such dosage forms are known, or will be apparent, to those skilled in this art; for example, see, Remington: The Science and Practice of Pharmacy, 21st Edition (Philadelphia College of Pharmacy and Science, 2005). The composition to be administered will, in any event, contain an effective amount of a chalcogenide composition to treat or prevent radiocontrast agent induced kidney injury.

In certain embodiments, the effective amount of a chalcogenide in a chalcogenide composition that is administered to a mammal is about, at least, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 mg, mg/kg, or mg/m², or any range derivable therein. Alternatively, the amount may be expressed as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 mM or M, or any range derivable therein.

In certain embodiments of the present invention, a chalcogenide composition is administered to a mammal for about, at least, at least about, or at most about 30 seconds, 1, 2, 3, 4, 5, 10, 15, 20, 25, 30, 35, 40, 45, 50, 55 minutes, 1, 2, 3, 4, 5, 6, 7, 8, 9, 10, 11, 12, 13, 14, 15, 16, 17, 18, 19, 20, 21, 22, 23, 24 hours, 1, 2, 3, 4, 5, 6, 7 days or more, and any range or combination therein.

Furthermore, when administration is intravenous, it is contemplated that the following parameters may be applied. A flow rate of about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 gtts/min or gtts/min, or any range derivable therein. In some embodiments, the amount of the solution is specified by volume, depending on the concentration of the compound of the present invention. An amount of time may be about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 minutes, 1, 2, 3, 4, 5, 6, 7, 8, 9, 10, 11, 12, 13, 14, 15, 16, 17, 18, 19, 20, 21, 22, 23, 24 hours, 1, 2, 3, 4, 5, 6, 7 days, 1, 2, 3, 4, 5 weeks, and/or 1, 2, 3, 4, 5, 6, 7, 8, 9, 10, 11, 12 months, or any range derivable therein.

Volumes of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 mls or liters, or any range therein, may be administered overall or in a single session.

The methods of the present invention may be practiced using a variety of different formulations of a chalcogenide composition, including both gas and liquid formulations. For example, in embodiments wherein the chalcogenide composition comprises sulfide, the sulfide may be administered as either a gas or a liquid. A variety of representative gaseous formulations of sulfide are described in, e.g., U.S. Patent Application Publication No. 2007/0078113, which is incorporated herein by reference, and a variety of representative liquid formulations of sulfide are described in U.S. Patent Application Publication Nos. 2008/0187604 and 2008/0199541, which are incorporated herein by reference. As one of skill in the art will appreciate, any of such gaseous and liquid formulations may be utilized in the present invention.

A gaseous formulation of hydrogen sulfide, or a pharmaceutically acceptable salt thereof, is provided in certain embodiments of the invention. With hydrogen sulfide gas, for example, in some embodiments, the concentration may be from about 0.01 to about 0.5 M (at STP). Typical levels of hydrogen sulfide contemplated for use in accordance with the present invention include values of about 1 to about 150 ppm, about 10 to about 140 ppm, about 20 to about 130 ppm, and about 40 to about 120 ppm, or the equivalent oral, intravenous or transdermal dosage thereof. Other relevant ranges include about 10 to about 80 ppm, about 20 to about 80 ppm, about 10 to about 70 ppm, about 20 to about 70 ppm, about 20 to about 60 ppm, and about 30 to about 60 ppm, or the equivalent oral, intravenous or transdermal thereof. It also is contemplated that, for a given mammal in a given time period, the sulfide atmosphere should be reduced to avoid a potentially lethal build up of sulfide in the subject. For example, an initial environmental concentration of 80 ppm may be reduced after 30 min to 60 ppm, followed by further reductions at 1 hr (40 ppm) and 2 hrs (20 ppm).

In other embodiments, a liquid chalcogenide composition is provided. In particular, a stable liquid pharmaceutical composition. Liquid pharmaceutical compositions of the present invention may include a chalcogenide composition in any desired concentration. The concentration may be readily optimized, e.g., depending upon the type of mammal being treated and the route of administration, so as to deliver an effective amount in a convenient manner and over an appropriate time-frame. In some embodiments, the concentration of chalcogenide, or pharmaceutically acceptable salt thereof, in the chalcogenide composition is in the range of 0.001 mM to 5,000 mM, in the range of 1 mM to 1000 mM, in the range of 50 to 500 mM, in the range of 75 to 250 mM, or in the range of 95 mM to 150 mM.

In embodiments wherein the liquid pharmaceutical compositions of the present invention comprise a chalcogenide composition comprising sulfide, the concentration of sulfide is in the range 1 to 250 mM. For example, in certain embodiments, the concentration of sulfide is in the range 10 to 200 mM.

In certain embodiments, the concentration of chalcogenide, or pharmaceutically acceptable salt thereof, in the chalcogenide composition is about, at least about, or at most about 0.001, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7. 3.8, 3.9, 4.0, 4.1, 4.2, 4.3, 4.4, 4.5, 4.6, 4.7, 4.8, 4.9, 5.0 mM or M or more or any range derivable therein (at standard temperature and pressure (STP)).

Molar concentration may be readily converted into weight per volume. Accordingly, any of the above ranges of molar concentration may be describe in terms of, e.g., mg/ml. Thus, in certain embodiments, the concentration of chalcogenide, or pharmaceutically acceptable salt thereof, in the chalcogenide composition is in the range of 0.01-1000 mg/ml, 0.1-100 mg/ml, or 1-10 mg/ml. In other embodiments, the concentration is approximately or is 1 mg/ml, 2 mg/ml, 3 mg/ml, 4 mg/ml, 5 mg/ml, 6 mg/ml, 7 mg/ml, 8 mg/ml, 9 mg/ml, or 10 mg/ml.

In certain aspects of the current invention, liquid pharmaceutical compositions comprise a chalcogenide composition dissolved in a liquid. In certain embodiments, the liquid is water (H₂O), while in other embodiments it is a more physiologically compatible solution such as phosphate-buffered saline (PBS) or Ringer's solution. In further embodiments, the liquid is sodium hydroxide in water, or potassium hydroxide in water. It is contemplated that in some embodiments, a liquid pharmaceutical composition is a saturated solution with respect to the chalcogenide composition.

In certain embodiments, the liquid pharmaceutical compositions of the present invention comprise sulfide measured at 80%-100% (w/v). In certain embodiments, liquid pharmaceutical compositions of the present invention comprise sulfide measured at 90%-100% (w/v). In certain embodiments, liquid pharmaceutical compositions of the present invention comprise sulfide measured at 95%-100% (w/v). In certain embodiments, liquid pharmaceutical compositions of the present invention comprise sulfide measured at 98%-100% (w/v).

In certain embodiments, the pH of a liquid pharmaceutical chalcogenide composition of the present invention is in the range of 3.0 to 12.0, while in other embodiments, the pH in the range of 5.0 to 9.0. The pH of the liquid pharmaceutical composition may be adjusted to a physiologically compatible range. For example, in certain embodiments, the pH of the liquid pharmaceutical composition is in the range of 6.5 to 8.5. In other embodiments, the liquid pharmaceutical compositions have a pH in the range of 7.5 to 8.5 or 7.4 to 9.0.

In certain embodiments, oxygen is measured in the range of 0 to 5 M in the pharmaceutical composition. In certain embodiments, oxygen is measured in the range of 0 to 3 M in the pharmaceutical composition. In certain embodiments, oxygen is measured in the range of 0.01 to 1M in the pharmaceutical composition. In certain embodiments, oxygen is measured at 0.001 to 1M in the pharmaceutical composition.

As noted above, the pharmaceutical compositions of the present invention may further comprise sulfide oxidation products. Oxidation products of the present invention include, but are not limited to, sulfite, sulfate, thiosulfate, polysulfides, dithionate, polythionate, and elemental sulfur. In various embodiments, one or more of these oxidation products is present in an amount less than 10%, less than 6.0%, less than 3.0%, less than 1.0%, less than 0.5%, less than 0.2%, less than 0.1%, less than 0.05%, or less than 0.01%.

In one embodiment, the oxidation product, sulfite, is present in the range of 0%-10% (w/v). In one embodiment, the oxidation product, sulfite, is in the range of 3.0%-6.0% (w/v). In one embodiment the oxidation product, sulfite, is in the range of 1.0%-3.0% (w/v). In one embodiment, the oxidation product, sulfite, is in the range of 0%-1.0% (w/v).

In one embodiment, the oxidation product, sulfate, is present in the range of 0%-10.0% (w/v). In one embodiment, the oxidation product, sulfate, is in the range of 3.0%-6.0% (w/v). In one embodiment, the oxidation product, sulfate, is in the range of 1%-3.0% (w/v). In one embodiment, the oxidation product, sulfate, is in the range of 0%-1.0% (w/v).

In one embodiment, the oxidation product, thiosulfate, is present in the range of 0%-10% (w/v). In another embodiment, the oxidation product, thiosulfate, is in the range of 3.0%-6.0% (w/v). In another embodiment, the oxidation product, thiosulfate, is in the range of 1.0%-3.0% (w/v). In another embodiment, the oxidation product, thiosulfate, is in the range of 0%-1.0% (w/v).

In one embodiment, the oxidation products include polysulfides present in the range of 0%-10% (w/v). In one embodiment, the oxidation products, polysulfides, are in the range of 3.0%-6.0% (w/v). In one embodiment the oxidation products, polysulfides, are in the range of 1.0%-3.0% (w/v). In one embodiment, the oxidation products, polysulfides, are in the range of 0%-1.0% (w/v).

In one embodiment, the oxidation product, dithionate, is present in the range of 0%-10% (w/v). In one embodiment, the oxidation product, dithionate, is in the range of 3.0%-6.0% (w/v). In one embodiment the oxidation product, dithionate, is in the range of 1.0%-3.0% (w/v). In one embodiment, the oxidation product, dithionate, in the range of 0%-1.0% (w/v).

In one embodiment, the oxidation product, polythionate, is present in the range of 0%-10% (w/v). In one embodiment, the oxidation product, polythionate, is in the range of 3.0%-6.0% (w/v). In one embodiment the oxidation product, polythionate, is in the range of 1.0%-3.0% (w/v). In one embodiment, the oxidation product, polythionate, is in the range of 0%-1.0% (w/v).

In one embodiment, the oxidation product, elemental sulfur, is present in the range of 0%-10% (w/v). In one embodiment, the oxidation product, elemental sulfur, is in the range of 3.0%-6.0% (w/v). In one embodiment the oxidation product, elemental sulfur, is in the range of 1.0%-3.0% (w/v). In one embodiment, the oxidation product, elemental sulfur, is present in the range of 0%-1.0% (w/v).

Those skilled in the art will recognize that a liquid pharmaceutical composition preferably remain stable during storage prior to administration to a mammal. In one embodiment, storage of the liquid pharmaceutical composition is about three months, and the storage temperature is in the range of 18° C.-27° C. In another embodiment, storage of the liquid pharmaceutical composition is about six months, and the storage temperature is in a range of 18° C.-27° C. In another embodiment, storage of the liquid pharmaceutical composition is about twelve months, and the storage temperature is in a range of 18° C.-27° C.

In one embodiment, storage of the liquid pharmaceutical composition is about three months, and the storage temperature is in a range of 4° C.-23° C. In another embodiment, storage of the liquid pharmaceutical composition is about six months, and the storage temperature is in a range of 4° C.-23° C. In another embodiment, storage of the liquid pharmaceutical composition is about twelve months, and the storage temperature is in a range of 4° C.-23° C.

In one embodiment, methods of preparing liquid pharmaceutical compositions of the present invention further comprise adjusting the osmolarity of the liquid pharmaceutical composition to an osmolarity in the range of 200-400 mOsmol/L.

In one embodiment, the osmolarity of the liquid pharmaceutical composition is in the range of 240-360 mOsmol/L or an isotonic range. In particular embodiments, the osmolarity of the liquid pharmaceutical composition is in the range of 250-330 mOsmol/L or the osmolality of the compositions is in the range of 250-350 mOsm/kg. NaCl may be used as an excipient to adjust osmolality.

In certain embodiments, isotonicity of liquid pharmaceutical compositions is desirable as it results in reduced pain upon administration and minimizes potential hemolytic effects associated with hypertonic or hypotonic compositions. Thus, the stabilized compositions of the invention not only have increased storage stability, but also have the added benefit of substantially reduced pain upon administration when compared with formulations using other more traditional buffer systems consisting of an acid and a salt form of the acid.

The following examples are provided for purposes of illustration, not limitation.

### EXAMPLES

### Example 1

### Kidney Protection Against Radiocontrast Agent Induced Nephropathy (RCIN)

Experimental Model for RCIN. Male Sprague Dawley rats weighing 300-350 g were used for the study. Rats were anesthetized by intraperitoneal injection of sodium pentobarbital, 50 mg/kg. The right jugular vein and left carotid artery were isolated and cannulated with PE50 tubings and heparinized blood sample, 0.4 ml, was collected from the venous catheter for baseline determination.

Systemic inhibition of prostaglandins and nitric oxide synthesis was accomplished by intravenous injection of indomethacin (10 mg/kg) followed by L-NAME (10 mg/kg) with 10 min apart. Ten minutes after L-NAME injection, kidney injury was induced by intra-arterial injection of radiocontrast agent (Renographin 60) at a dose of 6 ml/kg followed by returning the animals to cage for overnight recovery without access to water. On the next day, venous blood samples were collected through the precannulated jugular catheter. Kidney functions and gluco-homeostasis were assessed by measuring blood urine nitrogen (BUN), creatinine and glucose using a veterinarian diagnostic instrument (VetScan VS2).

Dosing Protocol. Bolus injection of vehicle or a liquid sulfide composition was administered intravenously 5 min before radiocontrast injection followed by continuous infusion of 180 min. The manufacture and formulation of liquid sulfide compositions suitable for use in the present invention are described in U.S. Patent Application Publication Nos. 2008/0187604 and 2008/0199541, which are incorporated herein by reference.

Study Groups.

Group 1: Vehicle: N=6.

Group 2: Liquid sulfide composition, 1 mg/kg bolus+2 mg/kg/hr infusion for 3 hrs, N=7.

Group 3: Liquid sulfide composition, 2 mg/kg bolus+4 mg/kg/hr infusion for 3 hrs, N=6.

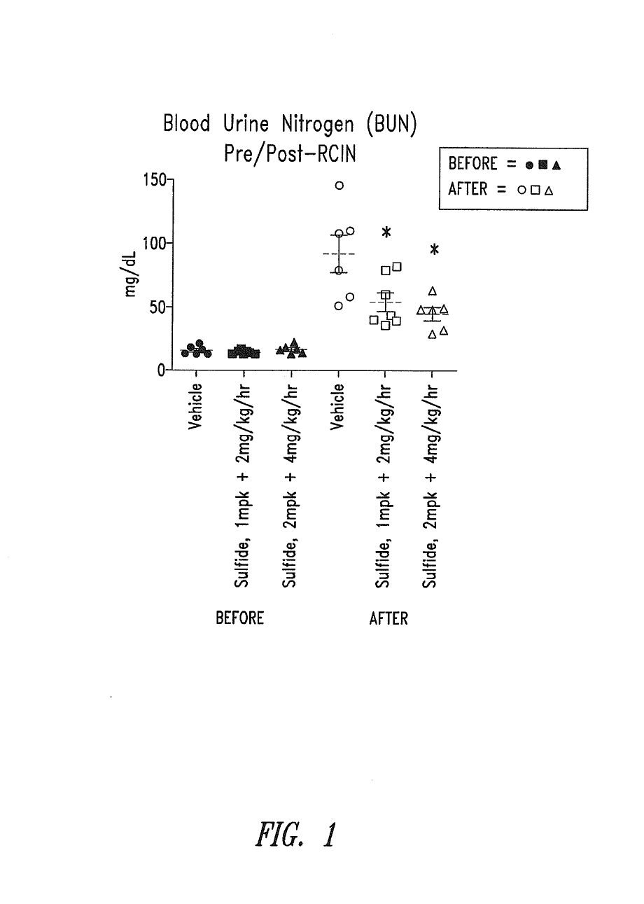

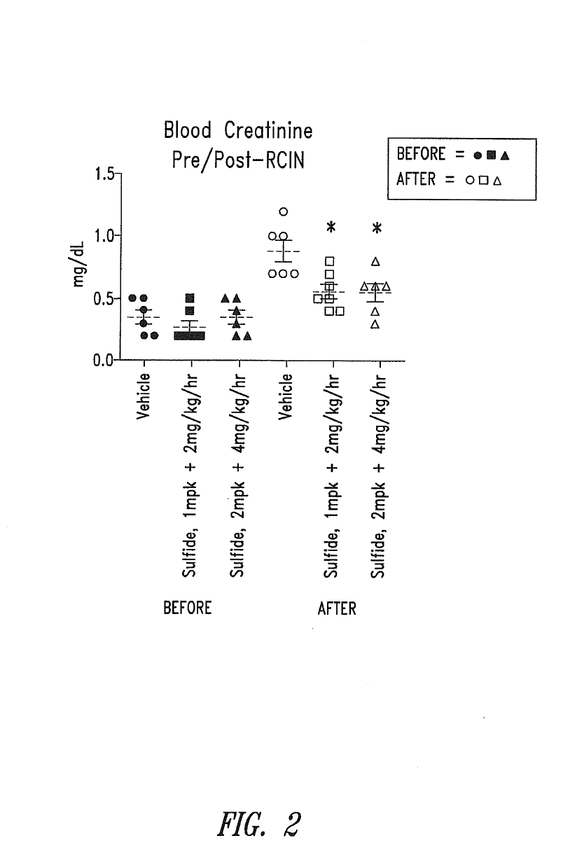

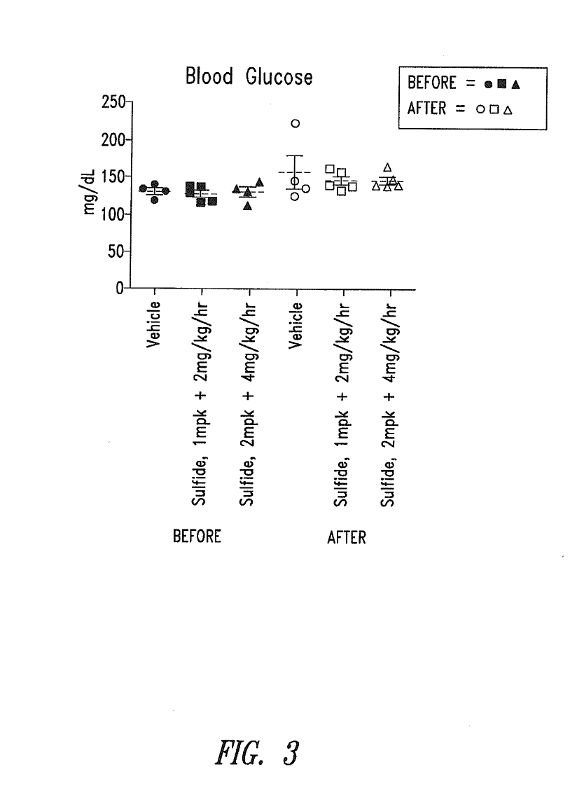

Results. As shown in FIG. 1, injection of radiocontrast agent increased BUN accumulation in blood and administration of the liquid sulfide composition significantly reduced the BUN accumulation. As shown in FIG. 2, injection of radiocontrast agent reduced renal excretion of creatinine and administration of the liquid sulfide composition significantly improved the renal function. As shown in FIG. 3, blood glucose was not changed by administration of the liquid sulfide composition, indicating no acute effect on flucose homeostasis.

### Example 2

### Methods of Manufacturing Representative Stable Liquid Sulfide Compositions

As noted above, representative stable liquid sulfide compositions suitable for use in Example 1 may be prepared as described in U.S. Patent Application Publication Nos. 2008/0187604 and 2008/0199541. For example, four representative stable liquid sulfide compositions may be prepared as follows.

Stock solutions are prepared using deoxygenated water. The water is deoxygenated by removing air under vacuum and dissolving with compressed nitrogen (99.99%) for 30 minutes. A saturated stock solution of 2.5 M Na₂S is prepared from Na₂S*9H₂O crystals (Fisher #5425) that are rinsed with oxygen-free, distilled, deionized water. This stock is stored tightly sealed and protected from light. A 220 mM stock solution of HCl is prepared by dilution of concentrated acid (Fisher # A144-212) and deoxygenated by dissolving with compressed nitrogen.

Liquid pharmaceutical compositions are prepared in a fume hood in a basic glove box filled with nitrogen gas to yield an oxygen-free environment. The reactor with pH meter, bubbler and stirrer are in the glove box. Oxygen levels in the glove box are monitored with an oxygen meter (Mettler-Toledo) with a sensitivity level of 0.03 μM. Methods of preparing the liquid pharmaceutical compositions include limiting oxygen content in each aspect of manufacturing and storage of the pharmaceutical composition where oxygen is measured in the range of 0 μM-5 μM in the pharmaceutical composition.

Liquid pharmaceutical compositions are prepared in a three-neck flask (Wilmad Labs) with each opening fitted with ground glass fittings having the following features.

A. A universal adapter with a plastic cap with a central orifice and O-ring. This adapter is fitted with a pH probe and sealed by the O-ring.

B. Universal adapter with a hose connector and a plastic cap with a central orifice and O-ring. This adapter is fitted with a gas dispersion tube with a glass frit. The dispersion tube is connected to a compressed gas cylinder and used to deoxygenate the solution by dissolving with compressed nitrogen and to neutralize the pH with a mixture of H₂S and nitrogen. The hose connector is fitted with a plastic tube to allow pressure to escape. These two connections are reversed to dispense the contents of the flask under positive nitrogen pressure.

C. The third neck is sealed with a ground glass stopper and used to add Na₂S solution or water to the flask.

Liquid Pharmaceutical Composition I—Na₂S nonahydrate. Liquid Pharmaceutical Composition I is prepared with the following steps.

a. Oxygen-free distilled, deionized water is added to a three neck flask and deoxygenated by dissolving with nitrogen for 30 minutes while stirring.

b. 2.5 M Na₂S stock is added to yield a 200 mM Na₂S solution.

c. The 200 mM Na₂S solution is bubbled with compressed nitrogen for 15 minutes while stirring.

d. 220 mM HCl is added until a final pH of 7.8-8.0 while dissolving with compressed nitrogen and stirring.

e. Deoxygenated deioinized water is added to give a final concentration of 100 mM Na₂S.

Liquid Pharmaceutical Composition II—Na₂S nonahydrate. Liquid Pharmaceutical Composition II is prepared with the following steps.

a. Deionized, oxygen-free water is added to the three neck flask and deoxygenated by dissolving with nitrogen for 30 minutes while stirring.

b. 2.5 M Na₂S stock is added to yield a 100 mM Na₂S solution.

c. The 100 mM Na₂S solution is bubbled with compressed nitrogen for 15 minutes while stirring.

d. The solution is bubbled with a 50/50 mixture of compressed nitrogen and CO₂ (99.9%) until a pH of 7.8 is reached.

Liquid Pharmaceutical Composition III—Na₂S with H₂S and Nitrogen.

Liquid Pharmaceutical Composition III is prepared with the following steps.

a. Deionized, oxygen-free water is added to the three neck flask and deoxygenated by dissolving with nitrogen for 30 minutes while stirring.

b. 2.5 M Na₂S stock is added to yield a 100 mM Na₂S solution.

c. The 100 mM Na₂S solution is bubbled with compressed nitrogen for 15 minutes while stirring.

d. The solution is bubbled with a 50/50 mixture of compressed nitrogen and H₂S until a pH of 8.2 is reached. This results in a final concentration of 90 mM sulfide.

Liquid Pharmaceutical Composition IV—H₂S. The final sulfide concentration of Liquid Pharmaceutical Composition IV is determined by the initial concentration of NaOH. Liquid Pharmaceutical Composition IV is prepared with the following steps.

a. NaOH in a range of 5 mM to 500 mM solution is added to the three neck flask with additives (DTPA, anti-oxidants).

b. The solution is deoxygenated by bubbling with argon at 5 psi for 15 minutes while stirring.

c. H₂S is bubbled through the solution while stirring until pH is reduced to 7.7 (or a range of 7.6 to 7.8).

d. The headspace in the flask is flushed with argon.

e. Amber dispensing bottles or vials are placed in a glove box that is flushed with a constant stream of argon and each bottle or vial is flushed with argon.

f. The formulation is dispensed under argon to maintain an oxygen-free environment.

The stability of the solution is monitored by measurement of sulfide concentration, pH, and absorbance spectrum (polysulfide formation). Additional assays are performed to monitor oxidation products which include sulfite, sulfate, thiosulfate, and elemental sulfur.

Liquid pharmaceutical compositions are dispensed within the sealed Glove box, from the three-necked flask under positive nitrogen pressure. Amber vials or amber bottles are filled to a slight over-pressure in an inert atmosphere argon or nitrogen to prevent/slow oxidative breakdown of the liquid pharmaceutical compositions, and sealed with plastic caps with Teflon/silicon liners or plastic caps with central Teflon lined silicon septa using a crown-cap crimper (Aldrich Z112976) to provide an air-tight seal.

All of the U.S. patents, U.S. patent application publications, U.S. patent applications, foreign patents, foreign patent applications and non-patent publications referred to in this specification are incorporated herein by reference, in their entirety to the extent not inconsistent with the present description.

From the foregoing it will be appreciated that, although specific embodiments of the invention have been described herein for purposes of illustration, various modifications may be made without deviating from the spirit and scope of the invention. Accordingly, the invention is not limited except as by the appended claims.

1. A method for treating or preventing radiocontrast agent induced kidney injury in a mammal comprising administering to the mammal a stable aqueous pharmaceutical composition comprising a first effective amount of HS⁻ and deoxygenated water prior to administering a radiocontrast agent to the mammal.

2. The method of claim 1, further comprising administering a second effective amount of HS⁻ to the mammal during administration of the radiocontrast agent to the mammal.

3. The method of claim 1, wherein the HS⁻ is made by dissolving in water a chalcogenide selected from H₂S, Na₂S, NaHS, K₂S, KHS, Rb₂S, CS₂S, (NH₄)₂S, (NH₄)HS, BeS, MgS, CaS, SrS, BaS, H₂Se, Na₂Se, NaHSe, K₂Se, KHSe, Rb₂Se, CS₂Se, (NH₄)₂Se, (NH₄)HSe, BeSe, MgSe, CaSe, SrSe, PoSe, BaSe, or, a pharmaceutically acceptable salt thereof.

4. The method of claim 3, wherein the HS⁻ is made by dissolving NaHS in water.

5. The method of claim 1, wherein the stable aqueous pharmaceutical composition comprises the HS⁻ and one or more pharmaceutically acceptable carriers, diluents and excipients.

6. The method of claim 5, wherein the first administration is a bolus injection, and, wherein the second administration is an intravenous infusion.

7. The method of claim 1, wherein the radiocontrast agent is administered before or during kidney diagnostic imaging.

8. The method of claim 1, wherein the radiocontrast agent is administered before or during heart blood vessel diagnostic imaging.

9. The method of claim 1, wherein the radiocontrast agent is an iodinated radiocontrast agent.

10. The method of claim 1, wherein the mammal is human.

11. The method of claim 1, wherein the mammal has a pre-existing impairment of renal function prior to administration of the radiocontrast agent.

12. The method of claim 10, wherein the human has a baseline creatinine level greater than or equal to 120 μmol/L, a glomerular filtration rate of less than 60 mL/min per 1.73 m² or a creatinine clearance of less than 60 mL/min.

13. The method of claim 10, wherein the human has one of more conditions selected from the group consisting of type 1 or 2 diabetes mellitus, atherosclerosis, congestive heart failure, an intraarterial balloon pump, anemia, a systolic blood pressure of less than 80 mm Hg, an age greater than 50 years, a glomerular filtration rate of less than 60 mL/min per 1.73 m² and reduced intravascular volume.

14. The method of claim 1, wherein the radiocontrast agent induced kidney injury is radiocontrast agent induced nephropathy or acute renal dysfunction.

15. The method of claim 1, wherein the deoxygenated water has an O₂ content ≦5 μM.

16. The method of claim 1, wherein the first and/or second effective amounts of HS⁻ are administered at a concentration in the range of 1 mM to 250 mM, or 10 mM to 200 mM.

17. The method of claim 1, including adjusting the stable aqueous pharmaceutical composition to a pH in the range of 6.5-8.5, 7.0-9.0, 7.5-8.5, 7.4-9.0, or, 7.5-8.0 by adding HCl or NaOH to the composition.

18. The method of claim 1, wherein the stable aqueous pharmaceutical composition further comprises one or more oxidation products selected from the group consisting of polysulfide, sulfite, sulfate and thiosulfate.

19. The method of claim 18, wherein said stable aqueous pharmaceutical compositions comprise polysulfide in the range of 0-1.0%, sulfite in the range of 0-1.0%, sulfate in the range of 0-1.0%, and, thiosulfate in the range of 0-1.0%.

20. The method of claim 1, wherein said composition has an osmolarity in the range of 250-330 mOsmol/L.

21. The method of claim 1, wherein the mammal is contacted with the pharmaceutical composition before or after the kidney injury.

22. The method of claim 1, wherein said composition is near isotonic.
